(19) United States
(12) Patent Application Publication
(10) Pub. No.: US 2024/0143908 A1
(43) Pub. Date: May 2, 2024

(54) METHOD FOR CREATING A TABLE FOR PROCESSING ATTRIBUTES OF COMPONENTS FROM A LABORATORY AND/OR PROCESS AREA

(71) Applicant: Analytik Jena GmbH, Jena (DE)

(72) Inventors: Nadine Taubert, Jena (DE); Vinh Van, Jena (DE); Heiko Oehme, Jena (DE); Andreas Schreiber, Schwäbisch Gmünd (DE); Julian Grüneberg, Schwäbisch Gmünd (DE)

(21) Appl. No.: 18/493,279

(22) Filed: Oct. 24, 2023

(30) Foreign Application Priority Data
Oct. 26, 2022 (DE) 10 2022 128 367.4

(51) Int. Cl. G06F 40/18; G16H 10/40

(52) U.S. Cl. CPC G06F 40/18; G16H 10/40

(57) ABSTRACT

A table for processing component attributes is provided. The table consists of cells, which form a matrix of columns and rows is provided. Each row is assigned to one component and contains information relating to the component. Each column is assigned to an attribute and contains attribute information. A first attribute is assigned to a first column, where the first attribute comprises a set of values, where the set includes all values of the first attribute for all components, where the method includes inserting a first value from the set of the first attribute into a first cell of a first row and column, and where the first value relates to the component assigned to the first row. The method also includes inserting a second value from the set of the first attribute into the first cell, where the second value relates to the component assigned to the first row.

### CROSS-REFERENCE TO RELATED APPLICATION

The present application is related to and claims the priority benefit of German Patent Application No. 10 2022 128 367.4, filed on Oct. 26, 2022, the entire contents of which are incorporated herein by reference.

### TECHNICAL FIELD

The present disclosure relates to a method for creating a table for processing attributes of components from a laboratory and/or process area, wherein the table consists of a plurality of cells, which form a matrix of columns and rows. The present disclosure furthermore relates to a computer program product for creating a table for processing attributes of components from a laboratory and/or process area, and to a computer-readable medium. Furthermore, the present disclosure relates to a method for processing attributes of components from a laboratory and/or process area by means of a table, and to a use of a table for processing the attributes of the components and/or planning processes in the laboratory and/or process area, and to a use of a table for monitoring the attributes of the components during the automated execution of a workflow.

### BACKGROUND

The process area comprises process plants and measuring points, which are generally monitored by means of field devices. Field devices serve, for example, for determining and/or monitoring the pressure, the pH value, the conductivity, and/or the flow rate. A sample can be taken from the process plant or measuring point or examined on-site, i.e., in the process. The laboratory area comprises laboratory devices to which samples are provided for examination. Laboratory devices are, for example, analysis devices which examine the samples, inter alia with regard to their composition and/or concentration.

A high throughput of measurements on samples is carried out daily in the laboratory and process area. This requires not only measuring and laboratory devices designed for this purpose, but also means for presenting and evaluating the measurement results. Due to the multiplicity of samples to be examined and the large number of available measurement methods, the presentation of the measurement results can quickly become unclear.

For example, a single sample can be examined with an entire series of analytical measurement methods, the measurement results of which are presented in the form of a table. In this case, it is generally too unclear to provide a separate column for each measurement method. Previous tables for presenting the measurement results therefore have subtables, which can be displayed and hidden by selecting a table element and in which the measurement results are displayed.

### SUMMARY

The object of the present disclosure is therefore to provide a method for creating a table for processing attributes of components from a laboratory and/or process area, which method generates a space-saving and clear table.

The object is achieved according to the present disclosure by a method for creating a table for processing attributes of components from a laboratory and/or process area according to claim 1, a computer program product for creating a table for processing attributes of components from a laboratory and/or process area according to claim 10, a computer-readable medium according to claim 11, a method for processing attributes of components from a laboratory and/or process area by means of a table according to claim 12, a use of a table for processing the attributes of the components and/or planning processes in the laboratory and/or process area according to claim 14, and a use of a table for monitoring the attributes of the components during the automated execution of a workflow according to claim 15.

The object is achieved according to the present disclosure by a method for creating a table for processing attributes of components from a laboratory and/or process area, where the table consists of a plurality of cells, which form a matrix of columns and rows. Each row is in each case assigned to one component and contains information relating to the respective component. Each column is assigned to an attribute and contains information relating to the attribute. A first attribute is assigned to a first column, where the first attribute comprises a set of values, where the set includes all values of the first attribute for all components.

The method includes a step of inserting a first value from the set of the first attribute into a first cell of a first row and the first column, where the first value relates to the component assigned to the first row. The method also includes a step of inserting at least a second value from the set of the first attribute into the first cell, where the second value relates to the component assigned to the first row.

Redundant columns are saved by assigning a set of values, which comprises all values of the first attribute for all components, to the first attribute and by inserting only the values relating to the respective component into the cells. If no set of values were assigned to the first attribute, the values would each have to be presented in a column, which would lead to a large number of columns. In addition, one or more cells of the columns could in each case remain empty, since a value of the column could not be assigned to each component. By combining the values of all components within the first attribute, only the first column is required, so that considerable space is saved within the table. The values of the attributes can be assigned to the components. An attribute is a generic term for information that can be assigned to the components.

Samples, laboratory devices, and/or measuring devices may be used as components. The samples can be examined in the laboratory and/or in the process. All types of devices that examine or analyze a sample by means of a suitable method may be referred to as laboratory and/or measuring devices. In addition, field devices such as are used, for example, in the process area are also referred to as measuring devices.

In one embodiment, the at least first and second values are represented in the form of tiles. Objects with a specifiable shape into which the at least first and second values are entered are referred to as tiles. The tiles may have rectangular, oval, or also other shapes.

In one development, the tiles are represented, highlighted, or framed by different colors. The tiles can be designed to be colored entirely or can have a colored frame.

In a further embodiment, a header and/or footer, which are arranged above or below the respective value and into which additional information relating to the respective value is inserted, is assigned to the tiles. By using the header and/or footer for additional information, less space is required in the table than if an additional cell were used for the additional information. This additional information is displayed within the respective tile (e.g., in the color-highlighted area of the tile).

A further embodiment provides that the first attribute comprise a set of parameters, wherein the respective parameter and, as a value, a value, e.g., a measured value, of the respective parameter are indicated in the header and/or footer. For example, the parameters are a temperature or a gas, at/in which a sample was examined. However, the parameters can also relate to laboratory and/or measuring devices which have been set by means of the parameters.

An alternative embodiment provides that the first attribute comprise a set of laboratory and/or process methods, wherein the respective laboratory and/or process method and, as a value, a measurement result of the respective laboratory and/or process method are indicated the header and/or footer. For example, a nitrogen content or a chlorine content measurement of a sample is indicated in the header and/or footer as a laboratory and/or process method, and, as a value, the content of nitrogen or chlorine in the sample determined in each case by the measurement.

In one development, laboratory and/or process methods, parameters, device statuses, process orders, workflows, measurement results, calibration information, statistical evaluations, quality control information, occupancy information of devices, and/or maintenance information are used as the first attribute. For example, the first attribute comprises a set of all available laboratory and/or process methods or measurement results. With regard to the maintenance information, the first attribute can in each case comprise, for example, at least one item of maintenance information for each component. With regard to the occupancy information of devices, the first attribute can comprise a defined number of positions of samples and/or containers containing the samples. With regard to the device status, the first attribute can comprise at least one item of information about the device status for each laboratory and/or process device. With regard to the quality control information, the first attribute can comprise at least one item of quality control information for each laboratory and/or process method. With regard to the statistical evaluations, the first attribute can comprise at least one item of statistical information for each laboratory and/or process method. With regard to the calibration information, the first attribute can comprise at least one calibration information for each measured value. With regard to the parameters, the first attribute can comprise at least one parameter for each laboratory and/or process method. With regard to the process orders and workflows, the first attribute can comprise a sample or several samples for each laboratory and/or process method.

In another embodiment, at least one of the at least first and second values is hidden, and a symbol is displayed instead, wherein the previously hidden value is displayed by selecting the symbol.

The objective aim is furthermore achieved according to the present disclosure by a computer program product for creating a table for processing attributes of components from a laboratory and/or process area, with computer-readable program code elements, which, when the computer program is executed by a computer, cause the computer to execute the method according to at least one of the preceding claims. Since, due to the first attribute, the created table requires less space than alternative solutions, the computer program product according to the present disclosure requires less computing power and storage capacity.

Furthermore, the object is achieved according to the present disclosure by a computer-readable medium on which at least the computer program according to claim 10 is stored.

The object is also achieved according to the present disclosure by a method for processing attributes of components from a laboratory and/or process area by means of a table. The table consists of a plurality of cells, which form a matrix of columns and rows. Each row is in each case assigned to one component and contains information relating to the respective component. Each column is assigned to an attribute and contains information relating to the attribute. A first attribute is assigned to a first column, wherein the first attribute comprises a set of values, wherein the set includes all values of the first attribute for all components,

The method includes inserting values into the table, where the values are provided in the components and/or in a superordinate unit, where, from the set of values, only those values that relate to the respective component are entered into the row of the respective component. The values inserted into the table are evaluated for at least one of the components using the table. Based upon a result of the evaluation, at least one measure is derived based upon a result of the evaluation for the at least one component.

Either the components themselves can provide the values to be inserted into the table, and/or the values are provided in a superordinate unit, such as a process control unit or a cloud, to which the components are connected. Because the first attribute in the first column includes all values of the first attribute for all components and only those values that relate to the respective component being inserted into the table, it is achieved that the table is kept as space-saving and clear as possible. By means of the space-saving table, the values assigned to the components can thus be evaluated in a simple manner. As a measure, it can be derived by means of the evaluation, for example, with which laboratory and/or process method a sample is to be examined in a next step.

In one development of the method according to the present disclosure, the values in the table are updated continuously or after specifiable time intervals.

Furthermore, the object is achieved according to the present disclosure by a use of a table for processing attributes of components in the laboratory and/or process area and/or for planning processes in the laboratory and/or process area, wherein the table consists of a plurality of cells, which form a matrix of columns and rows.

Each row is in each case assigned to one component and contains information relating to the respective component, and each column is assigned to an attribute and contains information relating to the attribute. A first attribute is assigned to a first column, where the first attribute comprises a set of values, and where the set includes all values of the first attribute for all components,

From the set of values, only those values that relate to the respective component are entered into the row of the respective component.

By means of the space-saving table, the attributes of the components can be processed in a simple manner by a computer program or a user. The table also allows simple planning of processes in the laboratory and/or process area, and, such as, for example, with regard to maintenance work and workflows.

Furthermore, the object is achieved according to the present disclosure by a use of a table for monitoring the attributes of the components during the automated execution of a workflow using values inserted into the table, wherein the table consists of a plurality of cells, which form a matrix of columns and rows.

Each row is in each case assigned to one component and contains information relating to the respective component. Each column is assigned to an attribute and contains information relating to the attribute.

A first attribute is assigned to a first column, where the first attribute comprises a set of values, where the set includes all values of the first attribute for all components. From the set of values, only those values that relate to the respective component are entered into the row of the respective component.

The space-saving table can also advantageously be used for monitoring attributes during the automated execution of a workflow. Thus, the values inserted into the table can be used to detect whether a set measurement has already been carried out, and the measurement results are entered in the table, or whether the desired parameters were used in the respective measurement. For example, progress or status information can also be displayed in the header or footer. Furthermore, the selection of suitable colors for the tiles also makes it possible to present information on the validity of the attributes. It is thus possible to highlight questionable or invalid measurement results with a signal color (e.g., red).

### BRIEF DESCRIPTION OF THE DRAWINGS

The present disclosure is explained in more detail below with reference to the following FIGS. 1-3. Shown are:

FIG. 1 shows a schematic representation of a table which is used in conjunction with the present disclosure.

FIG. 2 shows a further schematic representation of a table which is used in conjunction with the present disclosure.

FIG. 3 shows an example of a table which is used in conjunction with the present disclosure.

### DETAILED DESCRIPTION

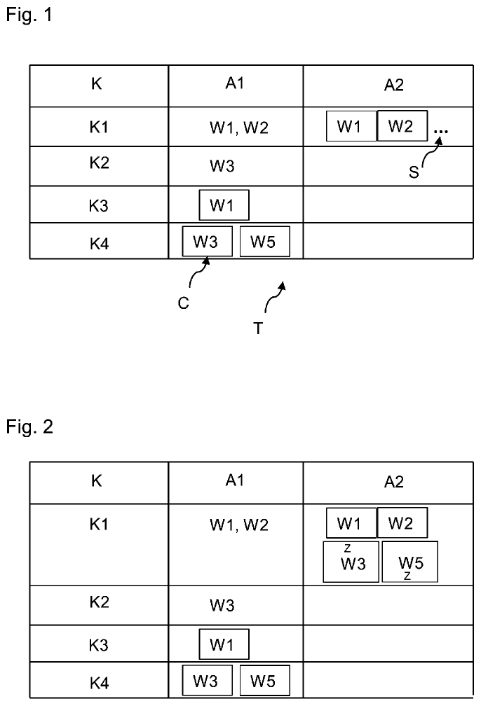

FIG. 1 shows an example of a table which is used in conjunction with the described methods and uses according to the present disclosure. The table consists of a plurality of cells which form a matrix of columns and rows. Each row is assigned to one component K1, K2, . . . and contains information relating to the respective component K1, K2, . . . . The columns are each assigned to an attribute An and contain information relating to the attribute An. The first column, which is the center column in FIG. 1, is assigned a first attribute A1; a second column, arranged on the right in FIG. 1, is assigned a second attribute A2. The first attribute A1 comprises a set of values Wn, which set includes all values of the first attribute A1 for all components K. In an analogous manner, the second attribute A2 in this case comprises, by way of example, a further set of values Wn.

According to the method according to the present disclosure for creating a table for processing attributes of components from a laboratory and/or process area, a first value W1 is inserted into the first cell of the first column and the first row, said first value relating to the component K1 assigned to the first row. In addition, a second value W2 is inserted into the same cell, which likewise relates to the component K1.

The first column associated with the first attribute A1 shows further values W3, W5, which are assigned to other components K2, K4. The set of values Wn assigned to the first attribute A1 can also comprise further values Wn. By assigning all values Wn to the first attribute A1, it is sufficient to insert into the first column only those values in the row of the respective component that are assigned to the respective component. If the values Wn were not assigned to the first attribute A1, a separate column would have to be generated for each value Wn, for example, so that the table would become very large and unclear. Alternatively, subtables that can be displayed as desired within or next to the table would have to be used. By contrast, the method according to the present disclosure creates a compact and clear table.

In FIG. 1, some of the values Wn inserted into the table are also shown in the form of tiles C. In this example, the tiles are rectangular and have a dark outline. Of course, other shapes of the colors are also conceivable. An optional symbol S in the form of three dots is also shown in the column assigned to the second attribute A2. By selecting the symbol S, previously hidden values Wn are displayed, as shown in FIG. 2. FIG. 2 is identical to FIG. 1 and shows the representation of the table after selecting the symbol S. By selecting the corresponding cell and/or the values Wn present in the cell, it is possible to change again into the representation of FIG. 1.

The previously hidden values W3, W5 in the column associated with the second attribute S2 in FIG. 2 also have a header and/or footer. The latter are each arranged above or below the value and provide additional information Z, which relates to the respective value.

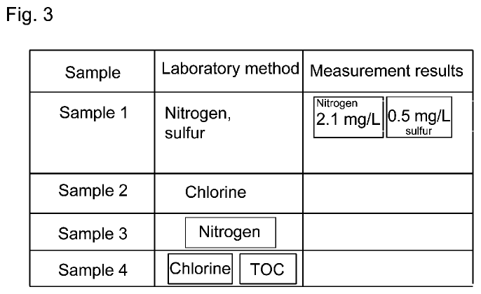

FIG. 3 shows a concrete exemplary embodiment of a table T, in which several samples 1-4 are assigned as attributes An to laboratory methods, applied to the sample, and the measurement results thereof. In this case, which laboratory method was applied to the respective sample and which is the measurement result determined in this case, insofar as an evaluation has already taken place, are inserted into the table. For example, the nitrogen content and sulfur content have already been determined for sample 1, and the chlorine content has already been determined for sample 2. The column with the measurement results indicates the measurement result determined by means of the method indicated in the header and/or footer.

With regard to the method according to the present disclosure for processing attributes An of components K from a laboratory and/or process area by means of a table T, a user can now evaluate the attributes An, i.e., the laboratory methods and the measurement results and, for example, the values thereof, and derive a measure for one of the samples. For example, the user may determine in the evaluation that the chlorine content for sample 2 has not yet been evaluated, and derive the measure to carry out the evaluation now. Alternatively, the user can evaluate the attributes for sample 3, and, as a measure, the still outstanding performance of the determination of the TOC content (total organic carbon content) must be determined.

According to the present disclosure, the table can be used to plan and monitor attributes An of components K in the laboratory and/or process area, and/or processes in the laboratory and/or process area. In this case, the table for monitoring the attributes can be viewed during each work step in the workflow and can, if necessary, be adjusted. For example, if it is detected that a series of samples delivers invalid results, the sequence of the samples to be measured can be adjusted in such a way that a different measurement series is first examined, in which possibly more advantageous measurement results can be obtained. The adjustment of the order of the measurement series can be carried out, for example, by moving the tiles in a cell.

1. A method for creating a table for processing attributes of components from a laboratory and/or process area,
wherein the table consists of a plurality of cells, which form a matrix of columns and rows;
wherein each row is in each case assigned to one component and contains information relating to the respective component;
wherein each column is assigned to an attribute and contains information relating to the attribute;
wherein a first attribute is assigned to a first column;
wherein the first attribute comprises a set of values, wherein the set includes all values of the first attribute for all components;
wherein the method has at least the following steps:
inserting a first value from the set of the first attribute into a first cell of a first row and the first column, wherein the first value relates to the component assigned to the first row; and
inserting at least a second value from the set of the first attribute into the first cell; wherein the second value relates to the component assigned to the first row.

2. The method of claim 1,
wherein samples, laboratory devices, and/or measuring devices are used as components.

3. The method of claim 1,
wherein the at least first value and the at least second value are presented in the form of tiles.

4. The method of claim 3,
wherein the tiles are represented, highlighted, or framed by different colors.

5. The method of claim 4,
wherein a header and/or footer, which are arranged above or below the respective value and into which additional information relating to the respective value is inserted, is assigned to the tiles.

6. The method of claim 5,
wherein the first attribute comprises a set of parameters, wherein the respective parameter and, as a value, a value of the respective parameter are indicated in the header and/or footer.

7. The method of claim 5,
wherein the first attribute comprises a set of laboratory and/or process methods, wherein the respective laboratory and/or process method and, as a value, a result of the respective laboratory and/or process method are indicated the header and/or footer.

8. The method of claim 1,
wherein laboratory and/or process methods, parameters, device statuses, process orders, workflows, measurement results, calibration information, statistical evaluations, quality control information, occupancy information of devices, and/or maintenance information are used as the first attribute.

9. The method of claim 1,
wherein at least one of the at least first and second value is hidden, and a symbol is displayed instead, wherein the previously hidden value is displayed by selecting the symbol.

10. A computer program product for creating a table for processing attributes of components from a laboratory and/or process area, with computer-readable program code elements, which, when the computer program is executed by a computer, cause the computer to execute the method according to claim 1.

11. The computer-readable medium of claim 10, wherein the computer program is stored on the computer-readable medium.

12. A method for processing attributes of components from a laboratory and/or process area by means of a table,
wherein the table consists of a plurality of cells, which form a matrix of columns and rows;
wherein each row is in each case assigned to one component and contains information relating to the respective component;
wherein each column is assigned to an attribute and contains information relating to the attribute;
wherein a first attribute is assigned to a first column, wherein the first attribute comprises a set of values, wherein the set includes all values of the first attribute for all components;
wherein the method has at least the following steps:
inserting values into the table, wherein the values are provided in the components and/or in a superordinate unit, wherein, from the set of values, only those values that relate to the respective component are entered into the row of the respective component,
evaluating the values, inserted into the table, for at least one of the components using the table, and
based upon a result of the evaluation, deriving at least one measure for the at least one component.

13. The method according to claim 14,
wherein the values in the table are updated continuously or after specifiable time intervals.

14. A use of a table for processing attributes of components in the laboratory and/or process area and/or for planning processes in the laboratory and/or process area,
wherein the table consists of a plurality of cells, which form a matrix of columns and rows;
wherein each row is in each case assigned to one component and contains information relating to the respective component;
wherein each column is assigned to an attribute and contains information relating to the attribute;
wherein a first attribute is assigned to a first column, wherein the first attribute comprises a set of values, wherein the set includes all values of the first attribute for all components, wherein, from the set of values, only those values that relate to the respective component are entered into the row of the respective component.

15. The use of a table for monitoring the attributes of components during automated execution of a workflow using values inserted into the table,
wherein the table consists of a plurality of cells, which form a matrix of columns and rows;
wherein each row is in each case assigned to one component and contains information relating to the respective component;
wherein each column is assigned to an attribute and contains information relating to the attribute;
wherein a first attribute is assigned to a first column;
wherein the first attribute comprises a set of values, wherein the set includes all values of the first attribute for all components;
wherein, from the set of values, only those values that relate to the respective component are entered into the row of the respective component.
